(19) United States
(12) Patent Application Publication
(10) Pub. No.: US 2026/0117185 A1
(43) Pub. Date: Apr. 30, 2026

(54) ENHANCEMENT OF FIBROBLAST PLASTICITY FOR TREATMENT OF DISC DEGENERATION

(71) Applicant: SPINALCYTE, LLC, Houston, TX (US)

(72) Inventors: Pete O'HEERON, Houston, TX (US); Thomas ICHIM, San Diego, CA (US)

(73) Assignee: SPINALCYTE, LLC, Houston, TX (US)

(21) Appl. No.: 18/736,046

(22) Filed: Jun. 6, 2024

Related U.S. Application Data
(63) Continuation of application No. 17/052,854, now abandoned.
(60) Provisional application No. 62/666,816, filed on May 4, 2018.

(51) Int. Cl. C12N 5/077; A61K 35/33; A61K 45/06

(52) U.S. Cl. CPC C12N 5/0656; A61K 35/33; A61K 45/06; C12N 2500/84; C12N 2501/065

(57) ABSTRACT

Embodiments of the disclosure include methods and compositions related to preparation of fibroblasts for use of treatment and prevention of a degenerative disc in an individual. In particular cases, fibroblasts are subject to de-differentiation that results in enhancement of their therapeutic activity and such methods include exposure of the fibroblasts to one or more agents and/or conditions.

This application is a continuation of U.S. patent application Ser. No. 17/052,854, filed Nov. 4, 2020, which is a national phase application under 35 U.S.C. § 371 of International Application No. PCT/US2019/030577, filed May 3, 2019, which claims priority to U.S. Provisional Patent Application Ser. No. 62/666,816, filed May 4, 2018, each of which are incorporated by reference herein in their entirety.

### TECHNICAL FIELD

Embodiments of the disclosure concern at least cell biology, molecular biology, biochemistry, and medicine.

### BACKGROUND

Generally speaking, the spine can be thought of as a column made of vertebrae and discs. The vertebrae provide the support and structure of the spine while the spinal discs, located between the vertebrae, act as cushions or “shock absorbers.” These discs also contribute to the flexibility and motion of the spinal column. As the body ages, the discs often develop deformities such as tears or cracks, or simply lose structural integrity, for example discs may bulge or flatten. These impaired discs can affect the anatomical functions of the vertebrae because of the resultant lack of proper biomechanical support and are often associated with chronic back pain. Disc degeneration may occur as part of the normal aging process or as a result of traumatic injury to the soft and flexible disc positioned between the vertebrae. The resulting structural collapse under load may cause, among other things, significant pain and loss of motion. Because of these conditions, other health issues may result. Several means of treating disc degenerative disease involve administration of regenerative cells into the disc as a source of regenerating atrophied or apoptotic cells in the nucleus pulposus of the disc. Unfortunately, current techniques for generating regenerative cells are limited. The disclosure provides means of generating cells useful for the treatment of disc degenerative disease.

### BRIEF SUMMARY

The present disclosure is directed to methods and compositions related to treatment and prevention of disc diseases in a mammalian individual, including disc degenerative disease. In particular embodiments, methods are disclosed that are directed to preparing fibroblasts for treatment and prevention of degenerative disc(s) in an individual. The fibroblasts are enhanced for such treatment and prevention methods by exposing them to one or more agents and/or one or more conditions such that the exposure enhances one or more capabilities and/or one or more activities of the treated cells.

In one embodiment, there is a method of preparing fibroblasts for use in treatment of a degenerative disc in an individual, comprising the step of exposing fibroblasts to one or more of the following de-differentiation agents: a) one or more histone deacetylase inhibitors; b) one or more DNA methyltransferase inhibitors; c) umbilical cord blood serum; d) one or more GSK-3 inhibitors; and/or e) one or more components from donor cells. In particular embodiments, the fibroblasts are exposed to reversin, cord blood serum, lithium, a GSK-3 inhibitor, resveratrol, pterostilbene, selenium, (−)-epigallocatechin-3-gallate (EGCG), valproic acid and/or salts of valproic acid, or a combination thereof. In some cases, the one or more components from the donor cells comprises RNA, DNA, protein, and/or cytoplasm from donor cells. When the agent is one or more components from donor cells, the fibroblasts may be cultured with one or more DNA demethylating agents, HDAC inhibitors, and/or histone modifiers. The fibroblasts may be further exposed to one or more proteolysis inhibitors, inhibitors of mRNA degradation, or both. Examples of proteolysis inhibitors include one or more protease inhibitors, proteasome inhibitors and/or lysosome inhibitors. The histone deacetylase inhibitor may be selected from the group consisting of a) valproic acid; b) sodium phenylbutyrate; c) butyrate; d) trichostatin A; and e) a combination thereof. In specific embodiments, the umbilical cord blood serum is used as part of culture media at a concentration of 0.1-20% volume/volume of the tissue culture media. The exposing step may occur in media having an oxygen content from 0.5 to 21%. The exposing step may occur in media having glucose content below 4.6 g/l.

In certain embodiments, an effective amount of the prepared fibroblasts are administered to an individual in need thereof. An effective amount of the prepared fibroblasts may be administered into the nucleus pulposus and/or the annulus fibrosus of the individual. The fibroblasts may be administered to the individual in or with a carrier, such as one that comprises one or more of beads, microspheres, nanospheres, hydrogels, gels, polymers, ceramics, and collagen platelet gels. The fibroblasts may be administered to the individual with (though not necessarily in the same composition) one or more additional therapeutic agents, such as one or more vitamins; nutritional supplements; hormones; glycoproteins; fibronectin; bone morphogenetic proteins (BMPs); differentiation factors; antibodies; gene therapy reagents; anti-cancer agents; genetically altered cells; and/or pain killers. In some cases, the fibroblasts are administered to the individual with one or more growth factors, although not necessarily in the same composition. In certain embodiments, the administration step may further comprise removal of at least some nucleus pulposus and/or annulus fibrosus of the individual.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### DETAILED DESCRIPTION

In reviewing the detailed disclosure that follows, and the specification more generally, it should be borne in mind that all patents, patent applications, patent publications, technical publications, scientific publications, and other references referenced herein are hereby incorporated by reference in this application, in their entirety to the extent not inconsistent with the teachings herein. It is important to an understanding of the present disclosure to note that all technical and scientific terms used herein, unless defined herein, are intended to have the same meaning as commonly understood by one of ordinary skill in the art. The techniques employed herein are also those that are known to one of ordinary skill in the art, unless stated otherwise.

For purposes of more clearly facilitating an understanding the invention as disclosed and claimed herein, the following definitions are provided.

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, oral ingestion, transdermal patch, topical, inhalation, suppository etc.

As used herein, “allogeneic” refers to tissues or cells from another body that in a natural setting are immunologically incompatible or capable of being immunologically incompatible, although from one or more individuals of the same species.

As used herein, the term “allotransplantation” refers to the transplantation of organs, tissues, and/or cells from a donor to a recipient, where the donor and recipient are different individuals, but of the same species. Tissue transplanted by such procedures is referred to as an allograft or allotransplant.

As used herein, the terms “allostimulatory” and “alloreactive” refer to stimulation and reaction of the immune system in response to an allologous antigens, or “alloantigens” or cells expressing a dissimilar HLA haplotype.

As used herein, “autologous” refers to tissues or cells that are derived or transferred from the same individual's body (i.e., autologous blood donation; an autologous bone marrow transplant).

As used herein, the term “autotransplantation” refers to the transplantation of organs, tissues, and/or cells from one part of the body in an individual to another part in the same individual, i.e., the donor and recipient are the same individual. Tissue transplanted by such “autologous” procedures is referred to as an autograft or autotransplant.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells that lack protein activity with an expression vector that expresses the protein, a derivative thereof, or a portion thereof. In other cases, a fragment of a gene product (such as a protein) may be considered biologically active (or it may be referred to as functionally active) if it retains the activity of the full-length gene product, although it may be at a reduced but detectable level of the activity of the full-length gene product.

“Cell culture” is an artificial in vitro system containing viable cells, whether quiescent, senescent or (actively) dividing. In a cell culture, cells are grown and maintained at an appropriate temperature, typically a temperature of 37° C. and under an atmosphere typically containing oxygen and CO₂, although in other cases these are altered. Culture conditions may vary widely for each cell type though, and variation of conditions for a particular cell type can result in different phenotypes being expressed. The most commonly varied factor in culture systems is the growth medium. Growth media can vary in concentration of nutrients, growth factors, and the presence of other components. The growth factors used to supplement media are often derived from animal blood, such as calf serum.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The term “drug”, “agent” or “compound” as used herein, refers to any pharmacologically active substance capable of being administered that achieves a desired effect. Drugs or compounds can be synthetic or naturally occurring, non-peptide, proteins or peptides, oligonucleotides, or nucleotides (DNA and/or RNA), polysaccharides or sugars.

The term “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce one or more symptoms of disc disease. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

### II. Enhancement of Fibroblast Plasticity and Compositions Thereof

Disclosed are novel means of endowing fibroblasts with enhanced plasticity so as to utilize the enhanced fibroblasts to promote treatment of disc degeneration in a mammal, including a human, dog, cat, horse, and so forth. The term “plasticity” means the ability of the cell to differentiate into other cells, and/or transdifferentiate into other cell types. In one embodiment, fibroblasts are endowed with an immature phenotype by exposure to one or more agents that induce a “cellular de-differentiation” so as to promote an enhanced ability of the fibroblasts to acquire at least chondrogenic and/or notochord characteristics. In one specific embodiment, fibroblasts are cultured in the presence of a de-differentiation cocktail comprising: a) a histone deacetylase inhibitor; b) a GSK-3 inhibitor; c) umbilical cord blood serum; or d) a combination thereof. The enhanced fibroblasts are then either administered to an individual (such as directly into a disc) for stimulation of disc regeneration, or in other embodiments are differentiated into the chondrocyte lineage, followed by delivery to the individual (such as by implantation).

The present disclosure provides means for changing the phenotype of fibroblast and/or fibroblastoid-like cells. In a specific embodiment, the disclosure provides means of “de-differentiating” fibroblasts to endow augmentation of plasticity, thus allowing for increased efficacy in generation of cells useful for therapeutic means. In particular aspects, the disclosure provides fibroblasts with augmented plasticity so as to increase therapeutic success when being delivered to an individual, such as injected into degenerating disc. By using one or more of epigenetic modifications, culture in human cord blood serum, or inhibition of GSK-3, the present disclosure allows for de-differentiation or transdifferentiation of cells for a recipient. In specific embodiments, cells originate from the individual that is in need of cell or gene therapy (autologous) or the cells originate from a donor (allogeneic). In the case of autologous use, methods of the disclosure solve the problem of immunorejection, as cells from one individual can be transformed into a different type of cell thereby allowing for the production or creation of specific types of cells needed for the treatment of a particular disease. Also, this disclosure provides for the formation from fibroblasts of donor de-differentiated cells, such as pluripotent cells, e.g., stem cells, thereby allowing for the derivation of different somatic cell phenotypes therefrom. In addition, while the cells produced according to the disclosure are useful for cell therapy they may also be used for study of mechanisms involved in cell differentiation and disease progression. In a particular embodiment, the cellular characteristics that are desired include the ability to generate proteoglycans and restore degenerated discs in individuals with intravertebral disc degenerative disease.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In a particular embodiment, fibroblasts are cultured in the cell culture system comprising a cell culture medium, such as in a culture vessel. In particular embodiments, a cell culture medium is supplemented with one or more agents and/or one or more conditions suitable for protecting the cells from in vitro aging and/or suitable for inducing in an unspecific or specific reprogramming. In a particular embodiment, an inducing substance according to the present disclosure is a substance selected from the group consisting of reversin, cord blood serum, lithium, a GSK-3 inhibitor, resveratrol, pterostilbene, selenium, a selenium-containing compound, (−)-epigallocatechin-3-gallate (EGCG), valproic acid and/or salts of valproic acid (such as sodium valproate), and a combination thereof.

In one embodiment of the present disclosure, reversin is utilized in methods. A concentration of reversin from 0.5 to 10 μM, such as of 1 μM, may be utilized in the cell culture. The concentration of reversin in the culture may be from 0.5-10, 0.5-9, 0.5-8, 0.5-7, 0.5-6, 0.5-5, 0.5-4, 0.5-3, 0.5-2, 0.5-1, 0.75-5, 0.75-4, 0.75-3, 0.75-2, 0.75-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 μM.

In one embodiment resveratrol is used in a culture of cells and a concentration may be used of 10 to 100 μM, such as 50 μM. The concentration may be in a range of 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 μM.

In one particular embodiment selenium and/or a selenium-containing compound are used in a culture, including at a concentration in the culture from 0.05 to 0.5 μM, such as 0.1 μM. The concentration of selenium or a selenium containing compound may be 0.05-0.5, 0.05-0.4, 0.05-0.3, 0.05-0.2, 0.05-0.1, 0.1-0.5, 0.1-0.4, 0.1-0.3, 0.1-0.2, 0.2-0.5, 0.2-0.4, 0.2-0.3, 0.3-0.5, 0.3-0.4, or 0.4-0.5 μM.

In another embodiment, cord blood serum is present in the tissue culture media, including at a concentration of 0.1%-20% volume to the volume of tissue culture media. The cord blood serum may be present at a concentration of 0.1-20, 0.1-15, 0.1-10, 0.1-5, 0.1-2.5, 0.1-1, 1-20, 1-15, 1-10, 1-5, 2.5-20, 2.5-15, 2.5-10, 2.5-5, 5-20, 5-15, 5-10, 10-20, or 15-20% volume of the volume of tissue culture media.

In one embodiment, the culture media comprises EGCG, including in a concentration from 0.001 to 0.1 μM, such as of 0.01 μM. the EGCG concentration in the culture media may be from 0.001 to 0.1, 0.001-0.05, 0.001-0.005, 0.005-0.1, or 0.005-0.05 μM.

In one embodiment valproic acid or sodium valproate is used in a culture, including in a culture in a concentration from 1 to 10 μM, such as 5 μM. The concentration of valproic acid or sodium valproate may be from 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3- 6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 μM.

Furthermore, in some fibroblast culture procedures, the cell culture medium may comprise, optionally in combination with one or more of the substances specified above, at least one proteolysis inhibitor, such as a transient proteolysis inhibitor. The use of at least one proteolysis inhibitor in the cell culture medium of the present disclosure increases the time the reprogramming proteins derived from the mRNA or any endogenous genes will be present in the cells and thus facilitates in an even more improved way the reprogramming by the transfected mRNA derived factors. The present disclosure uses, in a particular embodiment, as a transient proteolysis inhibitor a protease inhibitor, a proteasome inhibitor and/or a lysosome inhibitor. In a particular embodiment, the proteosome inhibitor is selected from the group consisting of MG132, TMC-95A, TS-341 and MG262. In one embodiment, the protease inhibitor is selected from the group consisting of aprotinin, G-64, leupeptin-hemisulfate, and a combination thereof. In a particular embodiment, the lysosomal inhibitor is ammonium chloride.

In one embodiment, a cell culture medium comprises at least one transient inhibitor of mRNA degradation. The use of at least a transient inhibitor of mRNA degradation increases the half-life of the reprogramming factors, in certain embodiments. Another embodiment of the present disclosure includes a condition suitable to allow translation of the transfected reprogramming mRNA molecules in the cells that includes an oxygen content in the cell culture medium from 0.5 to 21%. The oxygen content in the cell culture may be from 0.5-21, 0.5-20, 0.5-19, 0.5-18, 0.5-17, 0.5-16, 0.5-15, 0.5-14, 0.5-13, 0.5-12, 0.5-11, 0.5-10, 0.5-9, 0.5-8, 0.5-7, 0.5-6, 0.5-5, 0.5-4, 0.5-3, 0.5-2, 0.5-1, 1-21, 1-20, 1-19, 1- 18, 1-17, 1-16, 1-15, 1-14, 1-13, 1-12, 1-11, 1-10, 1-9, 1-8, 1-7, 1-6, 1-5, 1-4, 1-3, 1-2, 5-21, 5-20, 5-15, 5-10, 5-7, 7-21, 7-15, 7-10, 10-21, 10-15, 15-21%, and so forth. More particular, and without wishing to be bound to the theory, oxygen is used to further induce or increase Oct4 by triggering Oct4 via Hifla, in these situations concentrations of oxygen lower than atmospheric concentration are used, and can be ranging from 0.1% to 10%. In such embodiments, concentrations of the oxygen may be 0.1-10, 0.-7.5, 0.1-5, 0.1-2.5, 0.1-1, 0.5-10, 0.5-7.5, 0.5-5, 0.5-2.5, 0.5-1, 1-10, 1-7.5, 1-5, 5-10, 5-7.5, 7.5-10%, and so forth.

In one embodiment, conditions that are suitable to support reprogramming of the cells by the mRNA molecules in the cells are selected; more particularly, these conditions require a temperature from 30 to 38° C., such as from 31 to 37° C., including from 32 to 36° C.

The temperature of a culture of any kind for any method of the disclosure may be 30, 31, 32, 33, 34, 35, 36, 37, or 38° C., including in a range from 30-38, 30-37, 30-36, 30-35, 30-34, 30-33, 30-32, 30-31, 31-38, 31-37, 31-36, 31-35, 31-34, 31-33, 31-32, 32-38, 32-37, 32-36, 32-35, 32-34, 32-33, 33-38, 33-37, 33-36, 33-35, 33-34, 34-38, 34-37, 34-36, 34-35, 35-38, 35-37, 35-36, 36-38, 36-37, or 37-38° C.

The glucose content of the medium may be below 4.6 g/l, such as below 4.5 g/l, such as below 4 g/l, such as below 3 g/l, such as below 2 g/I and including 1 g/l. DMEM media containing 1 g/l glucose may be utilized and is commercially available as “DMEM low glucose” from companies such as PAA, Omega Scientific, Perbio and Biosera. More particular, and without wishing to be bound to the theory, high glucose conditions adversely support aging of cells (methylation, epigenetics) in vitro that may render the reprogramming difficult. In one embodiment of the present disclosure, the cell culture medium comprises glucose in a concentration from 0.1 g/l to 4.6 g/l, such as from 0.5 g/l to 4.5 g/l and including from 1 g/l to 4 g/l. The cell culture medium may comprise glucose in a concentration from 0.1-4.6, 0.1-4.5, 0.1-3, 0.1-2, 0.1-1, 1-4.6, 1-4, 1-3, 1-2, 2-4.5, 2-4, 2-3, 3-4.5, 3-4, 4-4.6 g/l, and so forth.

In accordance with the present disclosure, the term “de-differentiation” refers to the process of a cell “going back” in developmental time. In this respect, a de-differentiated cell acquires one or more characteristics previously possessed by that cell at an earlier developmental time point. An example of de-differentiation is the temporal loss of epithelial cell characteristics during wounding and healing. De-differentiation can occur in degrees. In the aforementioned example of wound healing, de-differentiation progresses only slightly before the cells re-differentiate to recognizable epithelia. A cell that has greatly de-differentiated, for example, is one that resembles a stem cell. De-differentiated cells can either remain de-differentiated and proliferate as a de-differentiated cell; they can re-differentiate along the same developmental pathway from which the cell had previously de-differentiated; or they can re-differentiate along a developmental pathway distinct from which the cell had previously dedifferentiated. Within the context of the present disclosure, a de-differentiated fibroblast possesses enhanced plasticity and ability to differentiate, or “re-differentiate” into other cells, including chondrocytes, notochord, or notochord-like cells.

In particular embodiments, the de-differentiated state of a treated cell or plurality thereof, which in the present disclosure is a fibroblast, can be verified, such as by increased expression of one or more genes, including one or more genes selected from the group consisting of alkaline phosphatase (ALP), OCT4, SOX2, human telomerase reverse transcriptase (hERT), SSEA-4, and a combination thereof. That is, in specific embodiments the somatic cells introduced with the re-programming gene(s) are treated with a functional peptide such as RGD or other agent, and then an initial process in which a colony is generated in the de-differentiation process the process may be observed through alkaline phosphatase staining (AP staining) that is a marker of stem cells; furthermore, expression of Oct4 may be verified by immunofluorescence (IF) using an Oct4 antibody. Finally, the MET (receptor for HGF) degree in the de-differentiation process of the somatic cells may be verified, such as by flow cytometry (FACS) using antibodies of THY1 as a marker of human dermal fibroblasts and an epithelial cell adhesion molecule (EPCAM) as a marker of the epithelial cell (as examples).

In accordance with the disclosure presented herein, the term “reprogramming” refers to remodeling, in particular erasing and/or remodeling, epigenetic markers of a cell such as DNA methylation, histone methylation and/or activating genes such an event may occur by inducing transcription factor signal systems, such as for Oct4. In particular, the reprogramming in the context of the present disclosure may include at least one de-differentiated and/or reprogrammed cell; in particular, it provides a cell having the characteristic of a multipotent cell, in particular pluripotent stem cell, for example. Thus, in case the cells to be reprogrammed are cells that already have a multipotent or pluripotent character, the present disclosure is able to maintain these cells by the reprogramming in their multi-or pluripotent state for a prolonged period of time. In case the cells to be reprogrammed are in an aged or differentiated state, the the encompassed methods of the disclosure allow the de-differentiation into a multipotent or pluripotent stem cell. In a particular embodiment, multipotent cells may be reprogrammed to become pluripotent cells. The cells of the disclosure are fibroblasts that are to be reprogrammed, in particular embodiments.

In accordance with the disclosure presented herein, the term “stem cell”, refers to any self-renewing pluripotent cell or multipotent cell or progenitor cell or precursor cell that is capable of differentiating into one or multiple cell types. Stem cells are thus cells able to differentiate into one or more than one cell type and have an unlimited growth potential, in specific embodiments. Stem cells include those that are capable of differentiating into cells of osteoblast lineage, a mesenchymal cell lineage (e.g. bone, cartilage, adipose, muscle, stroma, including hematopoietic supportive stroma, and tendon). “Differentiate” or “differentiation”, as used herein, refers to the process by which precursor or progenitor cells (i.e., stem cells) differentiate into specific cell types, e.g., osteoblasts. Differentiated cells can be identified by their patterns of gene expression and cell surface protein expression. “De-differentiate” or “de-differentiation”, as used herein, refers to the process by which lineage-committed cells (e.g., myoblasts or osteoblasts) reverse their lineage commitment and become precursor or progenitor cells (i.e., multipotent or pluripotent stem cells). De-differentiated cells can, for instance, be identified by changes of patterns of gene expression and/or cell surface protein expression associated with the lineage committed cells.

In accordance with the disclosure presented herein, the terms “cell culture” and “culturing of cells” refer to the maintenance and propagation of cells and preferably human, human-derived and animal cells in vitro.

In accordance with the disclosure presented herein, the term “cell culture medium” is used for the maintenance of cells in culture in vitro. For some cell types, the medium may also be sufficient to support the proliferation of the cells in culture. A medium according to the present disclosure comprises nutrients such as energy sources, amino acids and inorganic ions. In some embodiments, a medium may comprise a dye like phenol red, sodium pyruvate, several vitamins, free fatty acids, antibiotics, anti-oxidants and/or trace elements. For culturing the fibroblasts, including fibroblasts that are de-differentiated into stem cells or stem cell-like cells according to the present disclosure, any standard medium such as Iscove's Modified Dulbecco's Media (IMDM), alpha-MEM, Dulbecco's Modified Eagle Media (DMEM), RPMI Media and McCoy's Medium is suitable before and/or during reprogramming. Once the cells have been reprogrammed, they can in a particular embodiment be cultured in embryonic stem cell medium.

In some embodiments, the cells are transfected. In accordance with the disclosure presented herein, the term “Transfection” refers to a method of gene delivery that introduces a foreign nucleotide sequences (e.g. DNA/RNA or protein molecules) into a cell such as by a viral or non-viral method. In particular embodiments according to the present disclosure, foreign DNA/RNA/proteins are introduced to a cell by transient transfection of an expression vector encoding a polypeptide of interest, whereby the foreign DNA/RNA/proteins is introduced but eliminated over time by the cell and during mitosis. By “transient transfection” is meant a method where the introduced expression vectors and the polypeptide encoded by the vector, are not permanently integrated into the genome of the host cell, or anywhere in the cell, and therefore may be eliminated from the host cell or its progeny over time. Proteins, polypeptides, or other compounds can also be delivered into a cell using transfection methods.

In accordance with the disclosure presented herein, the embodiment of identifying a “sufficient period of time” to allow stable expression of the at least one gene regulator (gene that induces reprogramming, such as Oct4) in absence of the reprogramming agent and the “sufficient period of time” in which the cell is to be maintained in culture conditions supporting the transformation of the desired cell is within the skill of those in the art. The sufficient or proper time period will vary according to various factors, including but not limited to, the particular type and epigenetic status of cells (e.g. the cell of the first type and the desired cell), the amount of starting material (e.g. the number of cells to be transformed), the amount and type of reprogramming agent(s), the gene regulator(s), the culture conditions, presence of compounds that speed up reprogramming (for example, compounds that increase cell cycle turnover, modify the epigenetic status, and/or enhance cell viability), etc. In various embodiments a sufficient period of time to allow a stable expression of the at least one gene regulator in absence of the reprogramming agent is about 1 day, about 2-4 days, about 4-7 days, about 1-2 weeks, about 2-3 weeks or about 3-4 weeks. In various embodiments the sufficient period of time in which the cells are to be maintained in culture conditions supporting the transformation of the desired cell and allow a stable expression of a plurality of secondary genes is about 1 day, about 2-4 days, about 4-7 days, or about 1-2 weeks, about 2-3 weeks, about 3-4 weeks, about 4-6 weeks or about 6-8 weeks. In preferred embodiments, at the end of the transformation period, the number of transformed desired cells is substantially equivalent or even higher than an amount of cells a first type provided at the beginning.

In specific embodiments, fibroblasts in addition to or as an alternative to being exposed to one or more agents and/or one or more conditions may or may not be exposed to one or more donor components from one or more other types of cells. In some embodiments, the methods of the disclosure allow the generation of cells that are fully compatible with an individual by the transfer of total RNA and/or DNA and/or cytoplasm (as examples) from one cell type (donor) into that of a recipient cell, e.g., a human fibroblast or keratinocyte or white blood cell or other cell which is readily available, easily isolated and expandable in culture. For example, the disclosure includes methods in which a skin biopsy is obtained from which primary fibroblasts (or any other cell that is easy to obtain e.g. white blood cells, keratinocytes, etc.) are isolated, optionally expanded in vitro and later transdifferentiated or de-differentiated into desired cell populations by RNA transfection. For example if these recipient somatic cells are to be converted into pluripotent cells, RNA may be isolated from embryonic stem cells, human or non-human PGC's, human or non-human teratocarcinoma cells, preimplantation embryos, or oocytes from human or non-human sources and used to convert these somatic cells into a less de-differentiated state, ideally into pluripotent cells that may be used to derive different human cell lineages. During this process, the methods of the disclosure provide means of “semi-dedifferentiating” the cells, for example the fibroblasts, so as to not generate completely pluripotent cells, which in some cases are associated with the risk of teratoma formation, but only de-differentiated to a degree to allow for enhanced therapeutic activity, particularly in cases such as disc degenerative diseases.

In one embodiment of the disclosure, it may be desired to augment the length of time the cell may be reprogrammed in vitro prior to administration to a mammal for the treatment of disease. Accordingly, the donor cell may be optionally modified by the transient transfection of a plasmid comprising an oncogene flanked by loxP sites for the Cre recombinase and containing a nucleic acid encoding the Cre recombinase under the control of an inducible promoter. The insertion of this plasmid results in the controlled immortalization of the cell. After the cell is reprogrammed into the desired cell-type and is ready to be administered to a mammal, the loxP-oncogene-loxP cassette may be removed from the plasmid by the induction of the Cre recombinase that causes site-specific recombination and loss of the cassette from the plasmid. Because of the removal of the cassette comprising the oncogene, the cell is no longer immortalized and may be administered to the mammal without causing the formation of a cancerous tumor.

Donor cells (from which one or more components are transferred to fibroblast cells) that are useful for the disclosure are dependent on the desired use of the generated cell. For example, in one embodiment, RNA or mRNA may be extracted to achieve pluripotency in the ‘target’ cells; the cells include by way of example: Human and/or Mouse Embryonic Stem cell, Human and/or Mouse Primordial Germ Cells, Mouse Teratocarcinoma cells, Mouse Embryonic-carcinoma cells, preimplantation embryos and oocytes from any species including human and vertebrates such as amphibians, fish, and mammals. Examples of recipient or target cells into which RNA or mRNA can be introduced to achieve pluripotency or transdifferentiation in the ‘target’ cells include by way of example primary fibroblasts. Various sources of fibroblasts may be used, depending on tissue and age. Examples of somatic cells that may be used as the donor cell for trans-differentiation include any cell type that is desired for cell therapies including by way of example hepatocytes, lymphocytes, beta cells, neural cells, cardiac cells, lung cells. The current disclosure encompasses de-differentiation of target cells using total RNA or mRNA. The mRNA or total RNA used to effect de-differentiation may be isolated from cells that are either pluripotent or that are capable of turning into pluripotent cells (oocyte). Examples thereof include by way of example Ntera cells, human or other ES cells, primordial germ cells, and blastocysts. Alternatively the RNA used to effect de-differentiation may comprise mRNA encoding specific transcription factors. The total RNA or mRNA's may be delivered into target cells by different methods including e.g., electroporation, liposomes, and mRNA injection. Target cells into which RNAs are introduced and that are to be de-differentiated according to the disclosure may be cultured in a medium comprising one or more constituents that facilitates transformation of cell phenotype. These constituents include by way of example epigenetic modifiers such as DNA demethylating agents, HDAC inhibitors, and/or histone modifiers; and/or cell cycle manipulation and pluripotent or tissue-specific promoting agents, such as helper cells that promote growth of pluripotent cells, growth factors, hormones, and bioactive molecules. Examples of DNA methylating agents include 5-azacytidine (5-aza), MNNG, 5-aza, N-methl-N′-nitro-N-nitrosoguanidine, temozolomide, procarbazine, etc. Examples of methylation inhibiting agents include decitabine, 5-azacytidine, hydralazine, procainamide, mitoxantrone, zebularine, 5-fluorodeoxycytidine, 5-fluorocytidine, anti-sense oligonucleotides against DNA methyltransferase, and/or other inhibitors of enzymes involved in the methylation of DNA. Examples of histone deacetylase (“HDAC”) inhibitors may be selected from a group consisting of hydroxamic acids, cyclic peptides, benzamides, short-chain fatty acids, depudecin, and a combination thereof. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and/or pyroxamide. Examples of cyclic peptides include, but are not limited to, trapoxin A, apicidin and/or FR901228. Examples of benzamides include but are not limited to MS-27-275. Examples of short-chain fatty acids include but are not limited to butyrates (e.g., butyric acid and/or phenylbutyrate (PB)) Other examples include CI-994 (acetyldinaline) and trichostatine. Particular examples of histone modifiers include PARP, the human enhancer of zeste, valproic acid, and/or trichostatine. Particular constituents that may be utilized in a particular media in order to facilitate RNA transformation and dedifferentiation of the RNA comprising target cells into pluripotent cells include trichostatine, valproic acid, zebularine and/or 5-aza. Target cells into which RNA is introduced are cultured for a sufficient time in media that promotes RNA transformation until dedifferentiated cells (pluripotent) cells are obtained.

In one embodiment, the resultant de-differentiated cells are used to produce desired cell types or remodeled cells that may be used for transplantation, for use in animal models such as animal disease models or animal models used in the study of potential therapeutics or these may be employed in in vitro models, e.g. in studies of factors or conditions that promote the differentiation of pluripotent cells into desired cell lineages. One embodiment of the disclosure includes introduction of total RNA or mRNAs from one cell type, such as a pluripotent or somatic cell, into a desired human somatic cell, such as a fibroblast, in order to de-differentiate or transdifferentiate such cell(s) into a pluripotent cell or a different somatic cell corresponding to the lineage of the cell from which the donor total RNA is derived. This may be sufficient to effect cell de-differentiation or transdifferentiation. In some instances this methodology may be combined with other methods and treatments involved in the epigenetic status of the recipient or target cell such as the exposure to DNA and histone demethylating agents, histone deacetylase inhibitors, and/or histone modifiers. This disclosure therefore includes methods of changing the fate or phenotype of cells. By using epigenetic modifications, the subject methods can de-differentiate or transdifferentiate cells. The disclosure solves the problem of immuno-rejection that is evident when incompatible cells/tissues are used for transplantation. Cells from one individual can be transformed into a different type of cell allowing for the derivation of cells needed for the treatment of a particular disease from which the individual is suffering. One of the types of cells that can be produced by methods of the disclosure is pluripotent stem cells. This disclosure also offers an opportunity to the research community to study the mechanisms involved in cell differentiation and disease progression.

In addition, the recipient cells may be cultured under different conditions that enhance reprogramming efficiency such as co-culture of the RNA transfected cells with other cell types, conditioned medias, and by the supplementation of the culture medium with other biological agents such as growth factors, hormones, vitamins, etc. that enhance growth and maintenance of the cultured cells.

In one embodiment, fibroblasts are treated with one or more “inhibitor(s) of DNA methylation”. This term refers to one or more agents that can inhibit DNA methylation. DNA methylation inhibitors have demonstrated the ability to restore suppressed gene expression. Suitable agents for inhibiting DNA methylation include, but are not limited to 5-azacytidine, 5-aza-2-deoxycytidine, 1-.beta.-D-arabinofuranosil-5-azacytosine, and dihydro-5-azacytidine, and zebularine (ZEB), BIX (histone lysine methyltransferase inhibitor), and RG108. Concentration of DNA methylation inhibitors, as well as duration of exposure, is dependent on ability to induce expansion of plasticity. For example, expansion of plasticity may be measured by ability of fibroblasts to differentiate into other tissues. In a particular embodiment, fibroblasts are utilized to differentiate into chondrocytes. Methods of differentiating fibroblasts in chondrocytes (or in some situations mesenchymal stems cells into chondrocytes), and assessment of differentiation are known in the art [1-3].

“Inhibitor of histone deacetylation” refers to one or more agents that prevents the removal of the acetyl groups from the lysine residues of histones that would otherwise lead to the formation of a condensed and transcriptionally silenced chromatin. Histone deacetylase inhibitors fall into several groups, including: (1) hydroxamic acids such as trichostatin (A) [4-7], (2) cyclic tetrapeptides, (3) benzamides, (4) electrophilic ketones, and (5) aliphatic acid group of compounds such as phenylbutyrate and valporic acid. Suitable agents to inhibit histone deacetylation include, but are not limited to, valporic acid (VPA) [8-19], phenylbutyrate and Trichostatin A (TSA). One example, in the area of mesenchymal stem cells, of valproic acid enhancing pluripotency and therapeutic properties is provided by Killer et al. (Stem Cell Res Therap, 2017 April 26; 8(1):100) who showed that culture of cells with valproic acid enhanced immune regulatory and metabolic properties of mesenchymal stem cells. The culture systems described, as well as means of assessment, are provided to allow one of skill in the art to have a starting point for the practice of methods of the present disclosure [20, 21]. Without being bound to theory, valproic acid in the context of the current disclosure may be useful to increasing in vitro proliferation of de-differentiated fibroblasts while preventing senescence-associated stress. For example, Zhai et al. showed that in an in vitro pre-mature senescence model, valproic acid treatment increased cell proliferation and inhibited apoptosis through the suppression of the p16/p21 pathway. In addition, valproic acid also inhibited the G2/M phase blockage derived from the senescence stress [22].

In some embodiments, small RNAs that act as small activating RNA (saRNA) that induce activation of OCT4 expression are applied to fibroblasts to induce de-differentiation. In some cases this is combined at least with histone deacetylase inhibitors and/or GSK3 inhibitors and/or DNA methyltransferase inhibitors, in order to induce a de-differentiated phenotype in the fibroblasts. Such fibroblasts can subsequently be used as a source of cells for differentiation into nucleus pulposus cells and/or notochord cells. Examples of small RNAs that act as small activating RNAs of the OCT4 promotor are known in the art [23-28].

In some embodiments, fibroblasts are transfected with miRNA and de-differentiated before differentiating into nucleus pulposus cells and/or chondrocytes. Fibroblasts may be obtained from companies such as Lonza and cultured, for example, in Knockout DMEM medium (Invitrogen, Life Technologies Ltd) containing 10% fetal bovine serum (PAA), 2 mM L-glutamine (Invitrogen, Life Technologies Ltd), 1× MEM non-essential amino acid solution, 1× Penicillin/Streptomycin (PAA) and β-mercaptoethanol (Sigma-Aldrich). Fibroblasts may be transduced using lentiviral particles containing hsa-miR-145-5p inhibitor (Genecopoeia) at MOI=40 in the presence of 5 μg/ml Polybrene (Sigma-Aldrich). Transduced cells were selected for Hygromycin resistance (50-75 μg/ml). For transient miR-145 inhibition, 1×10⁵ fibroblasts are transfected with 100 pmoles miR-145 mirVana® miRNA inhibitor (Life Technologies Ltd) using Neon transfection system (Invitrogen). Transfection is carried out by two 1600 V pulses for 20 ms. For reprogramming, cells are transduced using CytoTune®-iPS Sendai Reprogramming Kit (Product number A1378001) (Life Technologies Ltd) according to manufacturer's instructions. The efficiency of fibroblast de-differentiation can be assessed by alkaline phosphatase (AP) activity staining using Alkaline Phosphatase Blue Substrate (Sigma-Aldrich) and by TRA-1-60 expression, as determined indirect immunofluorescence. Cells are washed with PBS, fixed by 4% paraformaldehyde for 10 minutes at room temperature, washed again with PBS, and incubated overnight at 4° C. with primary antibody against TRA-1-60 (MAB4360, Merck Millipore). Then cells are washed three times with PBS and incubated with Alexa 488-conjugated secondary antibody and observed under fluorescent microscope [29].

In one embodiment of the disclosure, fibroblasts are administered to a subject by any suitable route, including by injection (such as intramuscular injection), including in hypoxic areas. Suitable routes include intravenous, subcutaneous, intrathecal, oral, intrarectal, intrathecal, intra-omentral, intraventricular, intrahepatic, and intrarenal.

In certain embodiments, fibroblasts may be derived from tissues comprising skin, heart, blood vessels, bone marrow, skeletal muscle, liver, pancreas, brain, adipose tissue, foreskin, placental, and/or umbilical cord. In specific embodiments, the fibroblasts are placental, fetal, neonatal or adult or mixtures thereof.

The number of administrations of cells to an individual will depend upon the factors described herein at least in part and may be optimized using routine methods in the art. In specific embodiments, a single administration is required. In other embodiments, a plurality of administration of cells is required. It should be appreciated that the system is subject to variables, such as the particular need of the individual, which may vary with time and circumstances, the rate of loss of the cellular activity as a result of loss of cells or activity of individual cells, and the like. Therefore, it is expected that each individual could be monitored for the proper dosage, and such practices of monitoring an individual are routine in the art.

### III. Examples of Methods of Use of Enhanced Fibroblasts

In particular embodiments, fibroblasts manipulated as described herein may be provided in effective amounts to an individual in need thereof, such as an individual with one or more degenerative discs. In some cases the fibroblasts are provided to an individual at risk for having a degenerative disc, such as an individual over the age of 40, 45, 50, 55, 60, or greater; an athlete or former athlete; an individual that performs or did perform manual labor; and so forth.

In one embodiment, treating an intervertebral disc degeneration condition comprises administering fibroblast cells (including fibroblast cells enhanced by methods of the disclosure) that are co-cultured with human umbilical cord tissue, and in some cases cells are injected to an intervertebral disc in an amount effective to treat the disease or condition. When umbilical cord tissue is utilized, in at least some cases the umbilical cord tissue is substantially free of blood. In specific embodiments, when the enhanced fibroblasts are co-cultured with umbilical cord tissue they may be capable of self-renewal and/or expansion in culture and/or are capable of differentiating. In specific embodiments, the culture utilizes L-valine for growth and/or are grown in at least about 5% oxygen. As a result of the co-culture, the fibroblast cells acquire, after co-culture, at least one or more of the following characteristics: a) express oxidized low density lipoprotein receptor 1, reticulon, chemokine receptor ligand 3, and/or granulocyte chemotactic protein 2; b) do not produce CD117, HLA-DR and/or telomerase; c) express alpha smooth muscle actin; d) express, relative to an unmanipulated human fibroblast, mesenchymal stem cell, or iliac crest bone marrow cell, increased levels of oxidized low density lipoprotein receptor 1, interleukin 8, and/or reticulon 1; e) express CD10, CD13, CD44, CD73, and/or CD90; and/or e) a combination thereof.

One of skill in the art will understand that there exists numerous alternative steps for facilitating cell reprogramming that may be applied to fibroblasts. These methods include the destabilizing of the cell's cytoskeletal structure (for example, by exposing the cell to cytochalasin B), loosening the chromatin structure of the cell (for example, by using agents such as 5 azacytidine (5-Aza) and/or Valproic acid (VPA) and/or DNA demethylator agents such as MBD2), transfecting the cell with one or more expression vector(s) comprising at least one nucleic acid encoding one or more de-differentiating factors (for example, OCT4, SOX-2, NANOG, and/or KLF), using an appropriate medium for the desired cell of a different type and an appropriate differentiation medium to induce de-differentiation of the fibroblast, inhibiting repressive pathways that negatively affects induction into commitment the desired cell of a different type, growing the cells on an appropriate substrate for the desired cell of a different type, and growing the cells in an environment that the desired cell of a different type (or “-like” cell) would be normally exposed to in vivo, such as the proper temperature, pH and/or low oxygen environment (for example about 2-5% O₂). In various embodiments, the disclosure encompasses these and other related methods and techniques for facilitating cell reprogramming/dedifferentiation.

A number of signaling molecules have been identified to regulate the differentiation of chondrocyte from mesenchymal progenitor cells to their terminal maturation of hypertrophic chondrocytes, including bone morphogenetic proteins (BMPs), SRY-related high-mobility group-box gene 9 (Sox9), parathyroid hormone-related peptide (PTHrP), Indian hedgehog (Ihh), fibroblast growth factor receptor 3 (FGFR3), and β-catenin [30]. Administration of one or more of these factors, such as by gene transfection, mRNA transfection, and/or protein transfection or exposure to dedifferentiation media, or cytoplasmic transfer, may be used to generate chondrocytes before implantation into degenerated disc.

In the practice of the methods encompassed by the disclosure, fibroblasts that have been de-differentiated may be either “re-differentiated” by in vitro culture to generate nucleus pulposus cells, nucleus pulposus-like cells, notochordal cells, and/or chondrocytes, or alternatively, they may be administered in an immature form with the purpose that they will either differentiate in vivo, or one can provide factors that will result in acceleration of disc regeneration. Regardless which type of cells are administered, the cells may be collected from culture and may undergo a centrifugation process so as to concentrate them in an form in which they are deliverable to the disc in a pellet form in suspension. In another embodiment, the cells are delivered using a carrier. The carrier can comprise, or can be selected from, the group consisting of beads, microspheres, nanospheres, hydrogels, gels, polymers, ceramics, collagen platelet gels, and a combination thereof. The carrier, in solid or fluid form, can carry the cells in several different ways. The cells can be embedded, encapsulated, suspended and/or attached to the surface of the carrier. In one embodiment, the carrier encapsulates the cells, provides nutrients, and protects the cells when they are delivered inside the disc. After a period of time inside the disc, the carrier degrades and releases the cells. Specific types of the various carriers are described below. Specific embodiments of the disclosure provide that the fibroblasts that have been de-differentiated are administered in a sustained release device (i.e., sustained delivery device). The administered formulation can comprise the sustained release device. The sustained release device may be adapted to remain within the disc for a prolonged period and slowly release the de-differentiated fibroblasts contained therein to the surrounding environment. This mode of delivery allows the de-differentiated fibroblasts to remain in therapeutically effective amounts within the disc for a prolonged period. One or more additional therapeutic agents can also be delivered by a sustained delivery device.

For the practice of methods of the disclosure, synthetic scaffolds, such as fumaric-acid based scaffolds, have been designed and tailored to allow for attraction of certain cells and to provide direction for the cells to differentiate in desired areas. The cells can also be embedded in the scaffold and then injected into the target area without affecting the viability or proliferation of the cells. After implantation of the fumaric-acid based scaffold, it degrades over time and no further surgery is necessary to remove the scaffold.

Carriers useful for the practice of the methods of the disclosure can also comprise hydrogels. The cells are encapsulated in the polymer chains of the hydrogel after gelation. Hydrogels can be delivered in a minimally invasive manner, such as injection to the target area. The hydrogel is also resorbed by the body. Hydrogel properties such as degradation time, cell adhesion behavior and spatial accumulation of extracellular matrix can be altered through chemical and processing modifications.

There exist numerous hydrogels suitable for use in the present disclosure including water-comprising gels, i.e., polymers characterized by hydrophilicity and insolubility in water. See, for instance, “Hydrogels”, pages 458-459, in Concise Encyclopedia of Polymer Science and Engineering, Eds. Mark et al., Wiley and Sons (1990), the disclosure of which is incorporated herein by reference in its entirety. Although their use is optional in the methods of the present disclosure, the inclusion of hydrogels can be highly advantageous because they tend to possess a number of desirable qualities. By virtue of their hydrophilic, water-containing nature, hydrogels can house viable cells, such as de-differentiated fibroblasts, and can assist with load bearing capabilities of the disc.

For the practice of the methods, in one embodiment, the hydrogel is a fine, powdery synthetic hydrogel. Suitable hydrogels exhibit an optimal combination of properties, such as compatibility with the matrix polymer of choice and biocompatability. The hydrogel can include any one or more of the following: polysaccharides, proteins, polyphosphazenes, poly(oxyethylene)-poly(oxypropylene) block polymers, poly(oxyethylene)-poly(oxypropylene) block polymers of ethylene diamine, poly(acrylic acids), poly(methacrylic acids), copolymers of acrylic acid and methacrylic acid, poly(vinyl acetate), and/or sulfonated polymers.

Another means of administering de-differentiated fibroblasts is to deliver them with a polymer. In general, these polymers are at least partially soluble in aqueous solutions, e.g., water, or aqueous alcohol solutions that have charged side groups or a monovalent ionic salt thereof. There are many examples of polymers with acidic side groups that can be reacted with cations, e.g., poly(phosphazenes), poly(acrylic acids), and poly(methacrylic acids). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

In accordance with the present methods, there is provided a method of treating degenerative disc disease in an intervertebral disc having a nucleus pulposus, comprising administering autologous de-differentiated fibroblasts, or re-differentiated cells into a degenerated intervertebral disc.

In one embodiment, the de-differentiated fibroblasts can be delivered into the disc space with at least one (an) additional therapeutic agent, such as an agent to aid in the proliferation and differentiation of the cells. There can be, for example, one additional therapeutic agent (i.e., a second therapeutic agent) or there can be multiple additional therapeutic agents (e.g., second and third therapeutic agents). The additional therapeutic agent may be delivered simultaneously with the de-differentiated fibroblasts. In another embodiment, the additional therapeutic agent is delivered after administering the de-differentiated fibroblasts to the disc. In yet another, the additional therapeutic agent is administered first, i.e., prior to administering the de-differentiated fibroblasts to the disc.

The same carrier may or may not be used to deliver the cells and the additional therapeutic agent. In some embodiments, the cells are located on the surface of the carrier and the additional therapeutic agent is placed inside the carrier. In other embodiments, the cells and the additional therapeutic agent may be delivered using different carriers.

Other additional therapeutic agents that may be added to the disc include, but are not limited to: vitamins and other nutritional supplements; hormones; glycoproteins; fibronectin; peptides and proteins; carbohydrates (simple and/or complex); proteoglycans; oligonucleotides (sense and/or antisense DNA and/or RNA); bone morphogenetic proteins (BMPs); differentiation factors; antibodies (for example, antibodies to infectious agents, tumors, drugs or hormones); gene therapy reagents; and anti-cancer agents. Genetically altered cells and/or other cells may also be included in the matrix. If desired, substances such as pain killers (i.e., analgesics) and narcotics may also be admixed with the carrier for delivery and release to the disc space.

In some embodiments, one or more growth factors are examples of additional therapeutic agents. As used herein, the term “growth factor” encompasses any cellular product that modulates the growth or differentiation of other cells, particularly connective tissue progenitor cells. The growth factors that may be used in accordance with the present methods include, but are not limited to, members of the fibroblast growth factor family, including acidic and basic fibroblast growth factor (FGF-1 and FGF-2) and FGF-4, members of the platelet-derived growth factor (PDGF) family, including PDGF-AB, PDGF-BB and PDGF-AA; EGFs, members of the insulin-like growth factor (IGF) family, including IGF-I and-II; the TGF-. beta. superfamily, including TGF-beta1, 2 and 3 (including MP-52), osteoid-inducing factor (OIF), angiogenin(s), endothelins, hepatocyte growth factor and keratinocyte growth factor; members of the bone morphogenetic proteins (BMPs) BMP-1, BMP-3, BMP-2, OP-1, BMP-2A, BMP-2B, BMP-4, BMP-7 and BMP-14; HBGF-1 and HBGF-2; growth differentiation factors (GDFs), members of the hedgehog family of proteins, including indian, sonic and desert hedgehog; ADMP-1; GDF-5; and members of the colony-stimulating factor (CSF) family, including CSF-1, G-CSF, and GM-CSF; and isoforms thereof. The growth factor can be autologous such as those included in platelet rich plasma or obtained commercially. In one embodiment, the growth factor is administered in an amount effective to repair disc tissue.

In some embodiments, the growth factor is selected from the group consisting of TGF-beta, bFGF, IGF-1, and a combination thereof. These growth factor(s) promote regeneration of the nucleus pulposus and/or stimulate proliferation and/or differentiation of dedifferentiated fibroblasts, as well as extracellular matrix secretion. In one embodiment, the growth factor is TGF-beta. In specific cases, TGF-beta may be administered in an amount of between about 10 ng/ml and about 5000 ng/ml, for example, between about 50 ng/ml and about 500 ng/ml, e.g., between about 100 ng/ml and about 300 ng/ml. In one embodiment, at least one of the additional therapeutic agents is TGF-beta1. In one embodiment, another additional therapeutic agent is FGF. In some embodiments, platelet concentrate is provided as an additional therapeutic agent. In one embodiment, the growth factors released by the platelets are present in an amount at least two-fold (e.g., four-fold) greater than the amount found in the blood from which the platelets were taken. In some embodiments, the platelet concentrate is autologous. In some embodiments, the platelet concentrate is platelet rich plasma (PRP). PRP is advantageous because it comprises growth factor(s) that can re-stimulate the growth of the ECM and because its fibrin matrix provides a suitable scaffold for new tissue growth.

In accordance with the present methods, there is provided a method of treating degenerative disc disease in an intervertebral disc having a nucleus pulposus, comprising: a) administering autologous and/or allogeneic de-differentiated fibroblasts into the degenerating disc; and optionally b) transdiscally administering at least one additional therapeutic agent into the degenerating disc. For the purposes of the present disclosure, “transdiscal administration” may include, but is not limited to; a) injecting a formulation into the nucleus pulposus of a degenerating disc, such as a relatively intact degenerating disc; b) injecting a formulation into the annulus fibrosus of a degenerating disc, such as a relatively intact degenerating disc; c) providing a formulation in a patch attached to an outer wall of the annulus fibrosus, d) providing a formulation in a depot at a location outside but closely adjacent to an outer wall of the annulus fibrosus (“trans-annular administration”); and e) providing the formulation in a depot at a location outside but closely adjacent to an endplate of an adjacent vertebral body (“trans-endplate administration”). Also in accordance with the present disclosure, there is provided a formulation for treating degenerative disc disease, comprising: a) autologous and/or allogeneic de-differentiated fibroblasts; and b) at least one additional therapeutic agent, wherein the formulation is present in an amount suitable for administration into a degenerating disc.

Also in accordance with the present methods, there is provided a device for delivering a formulation for treating degenerative disc disease to the disc comprising: a) a chamber containing the formulation comprising de-differentiated allogeneic and/or autologous fibroblasts and at least one additional therapeutic agent; and b) a delivery port in fluid communication with the chamber and adapted to administer the formulation to the disc.

In some embodiments, the cells may be introduced (i.e., administered) into the nucleus pulposus and/or the annulus fibrosus depending on which extra-cellular matrix needs rebuilding. In other embodiments, the cells may be introduced into both regions of the disc. Specific therapeutic agents may be selected depending on the region of the disc where the cells are going to be delivered.

In some embodiments, the cells alone are administered (e.g., injected) into the disc through a needle, such as a small bore needle. In some embodiments, the needle has a bore of about 22 gauge or less, so that the possibilities of producing a herniation are mitigated. For example, the needle can have a bore of about 24 gauge or less, so that the possibilities of producing a herniation are even further mitigated.

If the volume of the direct injection of the cells or formulation is sufficiently high so as to cause a concern of over-pressurizing the nucleus pulposus, then in some cases at least a portion of the nucleus pulposus be removed prior to administration (i.e., direct injection) of the de-differentiated autologous and/or allogeneic fibroblasts. In some embodiments, the volume of removed nucleus pulposus is substantially similar to the volume of the formulation to be injected. For example, the volume of removed nucleus pulposus can be within about 80-120% of the volume of the formulation to be injected. In addition, this procedure has the added benefit of at least partially removing some degenerated disc from the patient.

When injecting the de-differentiated fibroblast cells into the nucleus pulposus, in some cases the volume of drug (i.e., formulation of cells suspended in growth medium or a carrier) delivered can be between about 0.5 ml and about 3.0 ml comprising cells suspended in growth medium or a carrier. When injected in these smaller quantities, it is believed that the added or replaced volume will not cause an appreciable pressure increase in the nucleus pulposus. Factors to consider when determining the volume of drug to be delivered include the size of the disc, the amount of disc removed and the concentration of the dedifferentiated cells in the growth medium or carrier.

In embodiments of the disclosure, there are methods of augmenting plasticity of a fibroblast for use in treatment of disc degenerative disease, the method comprising the steps of a) exposure of the fibroblast to one or more histone deacetylase inhibitors; b) exposure of fibroblasts to one or more DNA methyltransferase inhibitors; and/or c) exposure of fibroblasts to umbilical cord blood serum. In specific embodiments, a histone deacetylase inhibitor(s) is selected from the group consisting of a) valproic acid; b) sodium phenylbutyrate; c) butyrate; d) trichostatin A; and e) a combination thereof. The histone deacetylase inhibitor(s) may be administered to the fibroblasts at a concentration and time period sufficient to allow for reduction of cellular senescence, which may be quantified by telomere length, by beta-galactosidase (senescent cells begin expression of beta-galactosidase), and/or by the ability to differentiate into chondrocytes. Examples of DNA methyltransferase inhibitors include 5 azacytidine. In specific embodiments, cord blood serum is used as part of the culture media at a concentration of about 0.1-20% volume/volume of tissue culture media.

### IV. Kits of the Disclosure

Any of the cellular and/or non-cellular compositions described herein or similar thereto may be comprised in a kit. In a non-limiting example, one or more reagents for use in methods for preparing cellular therapy may be comprised in a kit. Such reagents may include cells; one or more histone deacetylase inhibitors; one or more DNA methyltransferase inhibitors; umbilical cord blood serum; one or more GSK-3 inhibitors; and/or one or more components from donor cells and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### REFERENCES

All publications mentioned in the specification are indicative of the level of those skilled in the art to which the invention pertains. All publications are herein incorporated by reference to the same extent as if each individual publication was specifically and individually indicated to be incorporated by reference.
1. Mao, Y., et al., Cell type-specific extracellular matrix guided the differentiation of human mesenchymal stem cells in 3D polymeric scaffolds. J Mater Sci Mater Med, 2017. 28(7): p. 100.
2. Pao, S. I., et al., Effect of microgravity on the mesenchymal stem cell characteristics of limbal fibroblasts. J Chin Med Assoc, 2017. 80(9): p. 595-607.
3. Suchorska, W. M., et al., Gene expression profile in human induced pluripotent stem cells: Chondrogenic differentiation in vitro, part A. Mol Med Rep, 2017. 15(5): p. 2387-2401.
4. Ghanjati, F. and S. Santourlidis, Effect on Multipotency and Phenotypic Transition of Unrestricted Somatic Stem Cells from Human Umbilical Cord Blood after Treatment with Epigenetic Agents. Stem Cells Int, 2016. 2016: p. 7643218.
5. Dai, X., et al., Somatic nucleus reprogramming is significantly improved by m-carboxycinnamic acid bishydroxamide, a histone deacetylase inhibitor. J Biol Chem, 2010. 285(40): p. 31002-10.
6. Durcova-Hills, G., et al., Reprogramming primordial germ cells into pluripotent stem cells. PLoS One, 2008. 3(10): p. e3531.
7. Huh, Y. H., J. H. Ryu, and J. S. Chun, Regulation of type II collagen expression by histone deacetylase in articular chondrocytes. J Biol Chem, 2007. 282(23): p. 17123-31.
8. Wang, Z., et al., Infusion of Valproic Acid Into the Renal Medulla Activates Stem Cell Population and Attenuates Salt-Sensitive Hypertension in Dahl S Rats. Cell Physiol Biochem, 2017. 42(3): p. 1264-1273.
9. Lim, J., et al., Valproic acid enforces the priming effect of sphingosine-1 phosphate on human mesenchymal stem cells. Int J Mol Med, 2017. 40(3): p. 739-747.
10. Luo, Z., et al., Effects of histone deacetylase inhibitors on regenerative cell responses in human dental pulp cells. Int Endod J, 2017.
11. Hawkins, K. E., et al., Human Amniocytes Are Receptive to Chemically Induced Reprogramming to Pluripotency. Mol Ther, 2017. 25(2): p. 427-442.
12. Mahapatra, P. S., et al., Valproic acid assisted reprogramming of fibroblasts for generation of pluripotent stem cells in buffalo (Bubalus bubalis). Int J Dev Biol, 2017. 61(1-2): p. 81-88.
13. Dziadosz, M., et al., Effects of Pharmacological Agents on Human Amniotic Fluid-Derived Stem Cells in Culture. Stem Cells Dev, 2016.
14. Dehghan, S., et al., Oct4 transcription factor in conjunction with valproic acid accelerates myelin repair in demyelinated optic chiasm in mice. Neuroscience, 2016. 318: p. 178-89.
15. Petkov, S., et al., Long-Term Culture of Porcine Induced Pluripotent Stem-Like Cells Under Feeder-Free Conditions in the Presence of Histone Deacetylase Inhibitors. Stem Cells Dev, 2016. 25(5): p. 386-94.
16. Chen, X., et al., Valproic Acid Enhances iPSC Induction From Human Bone Marrow-Derived Cells Through the Suppression of Reprogramming-Induced Senescence. J Cell Physiol, 2016. 231(8): p. 1719-27.
17. Shi, H., et al., Roles of p53 and ASFIA in the Reprogramming of Sheep Kidney Cells to Pluripotent Cells. Cell Reprogram, 2015. 17(6): p. 441-52.
18. Gajzer, D., et al., Epigenetic and molecular signatures of cord blood CD34(+) cells treated with histone deacetylase inhibitors. Vox Sang, 2016. 110(1): p. 79-89.
19. Dehghan, S., et al., Exogenous Oct4 in combination with valproic acid increased neural progenitor markers: an approach for enhancing the repair potential of the brain. Life Sci, 2015. 122: p. 108-15.
20. Killer, M. C., et al., Immunosuppressive capacity of mesenchymal stem cells correlates with metabolic activity and can be enhanced by valproic acid. Stem Cell Res Ther, 2017. 8(1): p. 100.
21. Fila-Danilow, A., et al., The influence of TSA and VPA on the in vitro differentiation of bone marrow mesenchymal stem cells into neuronal lineage cells: Gene expression studies. Postepy Hig Med Dosw (Online), 2017. 71(0): p. 236-242.
22. Zhai, Y., et al., Histone deacetylase inhibitor valproic acid promotes the induction of pluripotency in mouse fibroblasts by suppressing reprogramming-induced senescence stress. Exp Cell Res, 2015. 337(1): p. 61-7.
23. Wang, J., et al., Identification of small activating RNAs that enhance endogenous OCT4 expression in human mesenchymal stem cells. Stem Cells Dev, 2015. 24(3): p. 345-53.
24. Wang, B. and Y. Hu, Promoter-Targeted Small Activating RNAs Alter Nucleosome Positioning. Adv Exp Med Biol, 2017. 983: p. 53-61.
25. Xu, C., et al., Long non-coding RNA GAS5 controls human embryonic stem cell self-renewal by maintaining NODAL signalling. Nat Commun, 2016. 7: p. 13287.
26. Hadjimichael, C., et al., MicroRNAs for Fine-Tuning of Mouse Embryonic Stem Cell Fate Decision through Regulation of TGF-beta Signaling. Stem Cell Reports, 2016. 6(3): p. 292-301.
27. Li, H. L., et al., miR-302 regulates pluripotency, teratoma formation and differentiation in stem cells via an AKTI/OCT4-dependent manner. Cell Death Dis, 2016. 7: p. e2078.
28. Sandmaier, S. E. and B. P. Telugu, MicroRNA-Mediated Reprogramming of Somatic Cells into Induced Pluripotent Stem Cells. Methods Mol Biol, 2015. 1330: p. 29-36.
29. Barta, T., et al., Brief Report: Inhibition of miR-145 Enhances Reprogramming of Human Dermal Fibroblasts to Induced Pluripotent Stem Cells. Stem Cells, 2016. 34(1): p. 246-51.
30. Li, J. and S. Dong, The Signaling Pathways Involved in Chondrocyte Differentiation and Hypertrophic Differentiation. Stem Cells Int, 2016. 2016: p. 2470351.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. An ex-vivo method of preparing fibroblasts, comprising culturing fibroblasts in the presence of a de-differentiation cocktail comprising a combination of:
a) a histone deacetylase (HDAC) inhibitor,
b) a GSK-3 inhibitor, and
c) umbilical cord blood serum.

2. The method of claim 1, wherein the HDAC inhibitor is selected from the group consisting of: a) valproic acid; b) sodium phenylbuterate; c) buterate; d) trichostatin A; and e) a combination thereof.

3. The method of claim 1, wherein GSK-3 inhibitor comprises lithium or salt thereof.

4. The method of claim 1, further comprising exposing the fibroblasts to one or more of the following de-differentiation agents:
a) a DNA methyltransferase inhibitor,
b) RNA, DNA, protein and/or cytoplasm from donor cells, wherein the donor cells are cells that are either pluripotent or that are capable of turning into pluripotent cells.

5. The method of claim 1, wherein the fibroblasts are further exposed to one or more proteolysis inhibitors, inhibitors of mRNA degradation, or both.

6. The method of claim 5, wherein the proteolysis inhibitor is a protease inhibitor, a proteasome inhibitor and/or a lysosome inhibitor.

7. (canceled)

8. The method of claim 1, wherein said umbilical cord blood serum is used as part of culture media at a concentration of 0.1-20% volume/volume of the tissue culture media.

9. The method of claim 1, wherein culturing fibroblasts comprises culture media having an oxygen content from 0.5 to 21%.

10. The method of any claim 1, wherein culturing fibroblasts comprises culture media having glucose content below 4.6 g/l.

11. A method of treatment comprising administering an effective amount of fibroblasts prepared according to claim 1 to an individual in need thereof.

12. The method of claim 11, wherein an effective amount of the prepared fibroblasts are administered into the nucleus pulposus and/or the annulus fibrosus of the individual.

13. The method of claim 11, wherein the fibroblasts are administered to the individual in or with a carrier.

14. The method of claim 13, wherein the carrier comprises one or more of beads, microspheres, nanospheres, hydrogels, gels, polymers, ceramics, and collagen platelet gels.

15. The method of claim 11, wherein the fibroblasts are administered to the individual with one or more additional therapeutic agents.

16. The method of claim 15, wherein the one or more therapeutic agent comprises one or more vitamins; nutritional supplements; hormones; glycoproteins; fibronectin; bone morphogenetic proteins (BMPs); differentiation factors; antibodies; gene therapy reagents; anti-cancer agents; genetically altered cells; and/or pain killers.

17. The method of claim 11, wherein the fibroblasts are administered to the individual with one or more growth factors.

18. The method of claim 11, wherein the administration step further comprises removal of at least some nucleus pulposus and/or annulus fibrosus of the individual.
